(19) United States
(12) Patent Application Publication
(10) Pub. No.: US 2025/0329727 A1
(43) Pub. Date: Oct. 23, 2025

(54) HIGH ENERGY DENSITY LITHIUM-ION ELECTROCHEMICAL CELL FOR IMPLANTABLE MEDICAL DEVICES

(71) Applicant: Medtronic, Inc., Minneapolis, MN (US)

(72) Inventors: Hui Ye, Maple Grove, MN (US); Prabhakar A. Tamirisa, Brooklyn Park, MN (US); Gaurav Jain, Edina, MN (US)

(21) Appl. No.: 19/182,708

(22) Filed: Apr. 18, 2025

Related U.S. Application Data
(60) Provisional application No. 63/636,988, filed on Apr. 22, 2024.

(51) Int. Cl. H01M 4/525; A61N 1/378; H01M 4/02; H01M 4/136; H01M 4/62; H01M 10/0525; H01M 10/0567

(52) U.S. Cl. CPC H01M 4/525; A61N 1/378; H01M 4/136; H01M 4/623; H01M 4/625; H01M 10/0567; H01M 2004/021; H01M 2004/027; H01M 2004/028; H01M 10/0525

(57) ABSTRACT

Rechargeable lithium-ion cells and implantable medical devices including the same are provided herein with improved energy density, upper recharge voltages, and long-term stability. Rechargeable lithium-ion cells provided include an LTO negative electrode having one or both of a high negative electrode area specific capacity and a high active material loading value and an LCO positive electrode having one or both of high LCO utilization and high density.

### RELATED APPLICATIONS

This application claims the benefit of U.S. Provisional Application Ser. No. 63/636,988, filed Apr. 22, 2024, the entire contents of each of which are incorporated herein by reference.

### SUMMARY

The present disclosure generally relates to the field of lithium-ion batteries or cells, and more specifically, lithium-ion cells with improved energy density and power density.

Lithium-ion batteries or cells include one or more positive electrodes, one or more negative electrodes, and an electrolyte material provided within a case or housing. Separators made from a porous polymer or other suitable material may also be provided between the positive and negative electrodes to prevent direct contact between adjacent electrodes, and electrolyte material penetrates through pores in this porous polymer.

Rechargeable lithium-ion batteries have become the primary power source for portable electronics. Long-term service life is an important characteristic for rechargeable lithium-ion batteries, especially in applications such as electrical vehicles and implantable medical devices, which often require 10 or more years of service life. Energy density and power density are two key attributes of a battery. Energy density is the amount of energy in the battery compared to the volume of the battery and may be expressed, for example, as milliwatt-hours per cubic centimeter (mWh/cm³). Power density is the time rate of energy transfer of which a battery is capable and may be expressed, for example, in watts per liter (W/L). Both higher energy density and higher power density are generally desirable. For smaller batteries—like those used in implantable medical devices—high energy density, high power density, reliability, and long service life are all important attributes.

Batteries with greater specific capacities tend to last longer in discharge and may allow for miniaturization, such as for use in small form factor devices. In general, the energy density of a primary cell (that is, a cell configured to be discharged once and then discarded or recycled) is greater than the energy density of a secondary cell (that is, a rechargeable cell configured to be cycled through charge and discharge cycles for repeated use). In many applications, rechargeable batteries are preferred over primary batteries for benefits such as improving the lifespan of a device and reducing waste. Rechargeable batteries are especially desirable where replacing a primary battery would be difficult or impossible, such as in implantable medical devices, where accessing the device to replace its battery (or the device itself) could require an in-patient procedure. However, rechargeable batteries in implantable medical devices are generally recharged frequently because of lower energy density and small form factor.

In general, there is a need for electrochemical cells with improved energy density, capacity retention, rate retention, and long-term stability. Such cells could improve the lives, comfort, and quality of care for patients living with or receiving implantable medical devices such as pacemakers, insulin pumps, cardioverter-defibrillators, drug delivery pumps, and neurostimulators.

Embodiments disclosed herein may include a rechargeable lithium-ion cell having a negative electrode with a negative electrode active material including Li₄Ti₅O₁₂ (LTO), a positive electrode with a positive electrode active material including LiCoO₂ (LCO), a separator between the negative electrode and the positive electrode, and an electrolyte material including from 0.5 percent to 5 percent by weight vinylene carbonate. The negative electrode has a negative electrode area specific capacity of 2 milliAmp-hours per square centimeter (mAh/cm²), between 1.5 mAh/cm² and 3 mAh/cm², or between 1 mAh/cm² and 15 mAh/cm². The positive electrode has a density of 3.9 grams per cubic centimeter (g/cm³), between 3.7 g/cm³ and 4 g/cm³, or between 3 g/cm³ and 4.2 g/cm³. The cell has a cell upper recharge voltage of 2.8 volts (V), between 2.6 V and 3 V, or between 2.6 V and 3.4 V.

Embodiments disclosed herein may further include a rechargeable lithium-ion cell having a negative electrode with a negative electrode active material including LTO, a positive electrode with a positive electrode active material including LCO, a separator between the negative electrode and the positive electrode, and an electrolyte material including from 0.5 percent to 5 percent by weight vinylene carbonate. The negative electrode has a negative electrode active material loading value of 10 milligrams per square centimeter (mg/cm²) or greater, between 6 mg/cm² and 60 mg/cm², or between 9 mg/cm² and 15 g/cm². The positive electrode has a LCO utilization rate of 130 milliAmp-hours per gram (mAh/g) or greater, between 120 mAh/g and 220 mAh/g, or between 140 mAh/g and 180 mAh/g. The cell has a cell upper recharge voltage of 2.8 volts (V), between 2.6 V and 3 V, or between 2.6 V and 3.4 V.

Embodiments disclosed herein may yet further include a rechargeable lithium-ion cell having a negative electrode with a negative electrode active material including LTO, a positive electrode with a positive electrode active material including LCO, a separator between the negative electrode and the positive electrode, and an electrolyte material including from 0.5 percent to 5 percent by weight vinylene carbonate. The negative electrode has a negative electrode area specific capacity of 2 milliAmp-hours per square centimeter (mAh/cm²), between 1.5 mAh/cm² and 3 mAh/cm², or between 1 mAh/cm² and 15 mAh/cm². The positive electrode has a LCO utilization rate of 130 milliAmp-hours per gram (mAh/g) or greater, between 120 mAh/g and 220 mAh/g, between 140 mAh/g and 180 mAh/g, or between 160 mAh/g and 170 mAh/g. The cell has a cell upper recharge voltage of 2.8 volts (V), between 2.6 V and 3 V, or between 2.6 V and 3.4 V.

The LCO of the positive electrode active material may include particles having a multi-modal size distribution. The LCO of the positive electrode active material may include particles having a first average particle size and particles having a second average particle size; and the first average particle size may be between 10 micrometers (um) and 30 um and the second average particle size may be between 2 um and 8 um. The LCO of the positive electrode active material may include particles having a first average particle size and particles having a second average particle size; and the second average particle size may be between 5% and 15% the size of the first average particle size. The positive electrode may further include a binder comprising polyvinylidene fluoride (PVDF). The positive electrode may further include a carbon conductive agent comprising carbon black, graphite, or a combination thereof. The positive electrode may have a porosity of 18%, between 15% and 25%, or between 12% and 35%. The positive electrode may have an LCO utilization of 130 milliAmp-hours per gram (mAh/g) or greater, between 120 mAh/g and 220 mAh/g, or between 140 mAh/g and 180 mAh/g. The negative electrode may have a thickness of 0.13 millimeters (mm), between 0.1 mm and 0.2 mm, or between 0.05 mm and 0.5 mm. The negative electrode may have an active material loading value of 10 mg/cm² or greater, between 6 mg/cm² and 60 mg/cm², or between 9 mg/cm² and 15 mg/cm². The negative electrode may have a porosity of 37%, between 20% and 50%, or between 31% and 43%. The negative electrode may have an LTO utilization of 170 mAh/g, between 150 mAh/g and 180 mAh/g, or between 165 mAh/g and 175 mAh/g. The negative electrode may have an LTO content of 93%, between 90% and 96%, or between 85% and 98%. The positive electrode may have an LCO content of 96%, between 95% and 97%, or between 90% and 99%. The positive electrode may have a positive electrode area specific capacity, and a N/P capacity ratio of the negative electrode area specific capacity to the positive electrode area specific capacity may be 0.75:1, between 0.65:1 and 0.85:1, or between 0.5:1 and 1:1. The cell may have an electrolyte content of 6 grams/Amp-hour (g/Ah), between 5 g/Ah and 7 g/Ah, or between 2 g/Ah and 10 g/Ah. The cell may be chargeable at a charge rate of C/0.5, between C/1 and C/0.25, or between C/100 and C/0.1. The negative electrode may have a negative electrode area specific capacity of 2 mAh/cm², between 1.5 mAh/cm² and 3 mAh/cm², or between 1 mAh/cm² and 15 mAh/cm². The positive electrode may have a LCO utilization rate of 130 mAh/g or greater, between 120 mAh/g and 220 mAh/g, between 140 mAh/g and 180 mAh/g, or between 160 mAh/g and 170 mAh/g. The positive electrode may have a density of 3.9 g/cm³, between 3.7 g/cm³ and 4 g/cm³, or between 3 g/cm³ and 4.2 g/cm³.

Embodiments disclosed herein may still further include an implantable medical device including the rechargeable lithium-ion cell of one or more embodiments described herein.

### BRIEF DESCRIPTION OF DRAWINGS

FIG. 1 is a diagrammatic representation of a portion of an example of a lithium-ion battery.

FIG. 2A is a diagrammatic cross-sectional view of a portion of an example of a battery or cell.

FIG. 2B is a diagrammatic cross-sectional view of a portion of an example of a battery or cell having negative electrode active material provided on both sides of the negative electrode current collector.

FIG. 3 is a schematic view of a system in the form of an implantable medical device implanted within a body or torso of a patient.

FIG. 4 is a graphical representation of the results of 75° C. accelerated daily rate cycling tests for illustrative cells according to FIGS. 1, 2A, and 2B.

FIG. 5A is a graphical comparison of the energy densities of comparative electrochemical cells to illustrative cells according to FIGS. 1, 2A, and 2B with high LTO area specific capacities.

FIG. 5B is a graphical representation of recharge rates for illustrative cells according to FIGS. 1 and 2 with high LTO area specific capacities.

FIG. 5C is a graphical representation of the results of 75° C. accelerated daily rate cycling tests for illustrative cells according to FIGS. 1, 2A, and 2B with high LTO area specific capacities.

FIG. 6A is a graphical comparison of the results of 75° C. accelerated daily rate cycling tests for illustrative electrochemical cells according to FIGS. 1, 2A, and 2B with electrolyte material including vinylene carbonate (VC) and electrochemical cells with comparative electrolyte materials.

FIG. 6B is a graphical comparison of swelling measured after formation of illustrative electrochemical cells according to FIGS. 1, 2A, and 2B having electrolyte material including VC and electrochemical cells with comparative electrolyte materials.

FIG. 7A is a graphical representation of the voltage discharge curve of an illustrative electrochemical cell according to FIGS. 1, 2A, and 2B with high LCO utilization and an electrolyte material including VC.

FIG. 7B is a graphical representation of charge rate before and after 16 weeks of 75° C. accelerated daily rate cycling testing for illustrative electrochemical cells according to FIGS. 1, 2A, and 2B with relatively high LCO utilization and an electrolyte material including VC.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various illustrative embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art, unless otherwise specified. The definitions provided herein are to facilitate understanding of certain terms used frequently herein and are not meant to limit the scope of the present disclosure.

Unless otherwise indicated, the terms “polymer,” “polymerized monomers,” and “polymeric material” include, but are not limited to, organic homopolymers, copolymers, such as, for example, block, graft, random and alternating copolymers, terpolymers, etc., and blends and modifications thereof. Furthermore, unless otherwise specifically limited, the term “polymer” shall include all possible geometrical configurations of components, such as repeating units, of the polymer material. These configurations include, but are not limited to, isotactic, syndiotactic, and atactic symmetries.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. and 10 or less includes 10, 9.4, 7.6, 5, 4.3, 2.9, 1.62, 0.3, etc.). Where a range of values is “up to,” “at most,” or “at least” a particular value, that value is included within the range.

As used herein, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Such inclusive or open-ended words encompass more restrictive terms or phrases, such as “consisting essentially,” or closed terms or phrases, such as “consisting”.

Reference in the specification to “some embodiments,” “an embodiment,” “one embodiment,” “one or more embodiments,” “embodiments,” “at least one embodiment”, or “other embodiments” means that a particular feature, structure, or characteristic described in connection with the embodiments is included in at least one or more embodiments, but not necessarily all embodiments, of the present disclosure. To the extent that the disclosure describes aspects, components, or elements associated with a particular embodiment in more detail or breadth, it is contemplated that the aspects, components, or elements associated with such embodiment should be understood to encompass the additional detail and breadth described in the disclosure.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the present disclosure.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

As described herein, lithium-ion batteries having high upper recharge voltages, dense positive electrodes, and thick negative electrodes provide improved characteristics, including greater long-term stability, power density, and energy density.

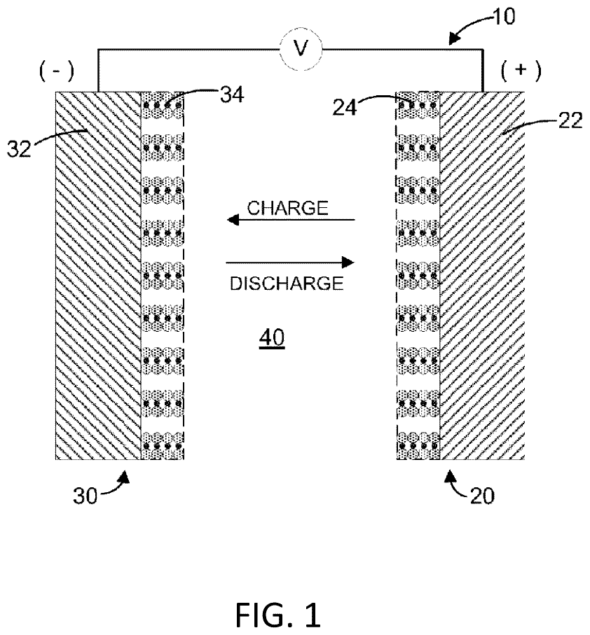

A diagrammatic representation of a portion of an illustrative lithium-ion battery 10 is shown in FIG. 1. The battery 10 includes a positive electrode 20 that includes a positive current collector 22 and a positive electrode active material 24, a negative electrode 30 that includes a negative current collector 32 and a negative electrode active material 34, an electrolyte material 40, and a separator (e.g., a polymeric microporous separator, not shown) provided intermediate, or between, the positive electrode 20 and the negative electrode 30. The electrodes 20, 30 may be provided as relatively flat or planar plates or may be wrapped or wound in a spiral or other configuration (e.g., an oval configuration). It will be understood in light of the present disclosure that any suitable electrochemical cell geometry or construction (such as rectangular, prismatic, cylindrical, oval, coiled, stacked, folded, etc.) may be used and the disclosure is not limited in this regard. The electrodes 20, 30 may also be provided in a folded configuration. The battery 10 may be at least partially disposed within (e.g., enclosed within) a housing (not shown).

During charging and discharging of the battery 10, lithium ions move between the positive electrode 20 and the negative electrode 30. For example, when the battery 10 is being discharged, lithium ions flow from the negative electrode 30 to the positive electrode 20. In contrast, when the battery 10 is being charged, lithium ions flow from the positive electrode 20 to the negative electrode 30.

Once assembly of the battery is complete, an initial charging operation (referred to as a “formation process”) may be performed. During the formation process, a stable solid-electrolyte inter-phase (SEI) layer is formed at the negative electrode and, in some cases, at the positive electrode. These SEI layers may act to passivate the electrode-electrolyte interfaces and may prevent side-reactions thereafter.

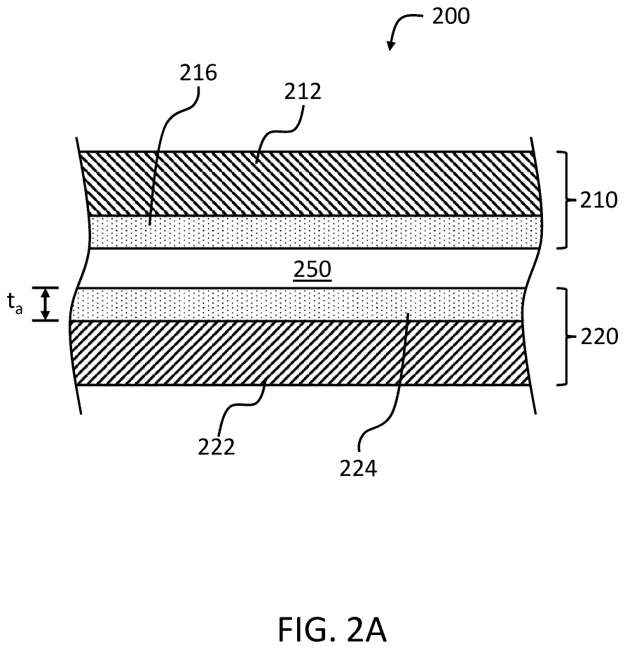

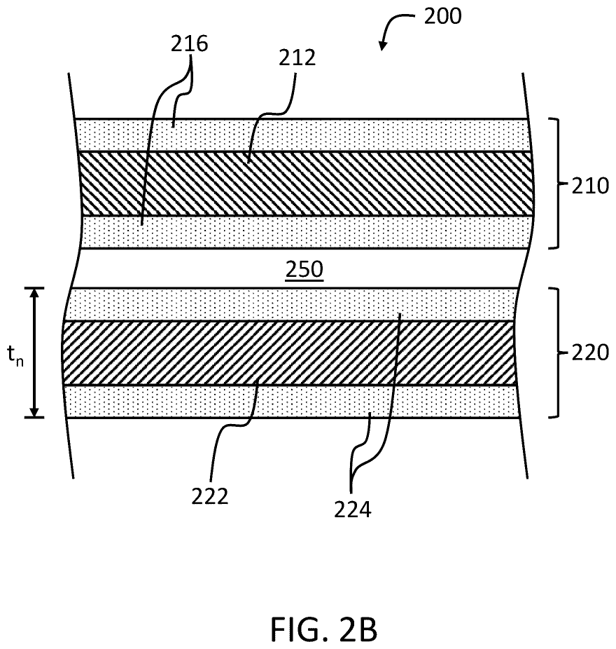

FIGS. 2A and 2B are schematic cross-sectional views of a portion of a battery or cell 200 according to illustrative embodiments that includes at least one positive electrode 210 and at least one negative electrode 220. The size, shape, and configuration of the battery may be selected based on the desired application or other considerations. For example, the electrodes may be flat plate electrodes, wound electrodes (e.g., in a jellyroll, folded, or other configuration), or folded electrodes (e.g., Z-fold electrodes). According to other illustrative embodiments, the battery may be a button cell battery, a thin film solid state battery, or another suitable type of lithium-ion battery.

A separator 250 is provided intermediate, or between, the positive electrode 210 and the negative electrode 220. The separator 250 may be, or may include, any suitable material or combination of materials. Suitable separator materials may include, for example, a polymeric material, such as a polypropylene/polyethylene copolymer or another polyolefin multilayer laminate including micropores formed therein to allow electrolyte lithium ions to flow from one side of the separator to the other.

The positive electrode 210 includes a positive electrode current collector 212. The positive electrode current collector 212 may be, or may include, any suitable material and, in particular, any suitable conductive material, such as titanium, a titanium alloy, aluminum, or an aluminum alloy. In some embodiments, the positive electrode current collector 212 may be, or may include, a foil, such as aluminum foil or an aluminum alloy foil. Positive electrode current collectors that are, or include, a foil may advantageously enable a greater ratio of positive electrode active material to total positive electrode material (e.g., by weight, by volume, etc.). Positive electrode current collectors that are, or include, aluminum/aluminum alloy may advantageously be relatively inexpensive, easily formed into a current collector, electrically conductive, readily weldable, corrosion resistant, and generally commercially available. Additionally, positive electrode current collectors that are, or include aluminum/aluminum alloy may advantageously have a relatively low density, which enables a greater ratio of positive electrode active material to total positive electrode material, particularly by weight.

The positive electrode 210 further includes a positive electrode active material 216 disposed (e.g., deposited, coated, etc.) on the positive electrode current collector 212. While FIG. 2 shows the positive electrode active material 216 provided on only one side of the positive electrode current collector 212, a layer of active material similar or identical to the positive electrode active material 216 may be provided (e.g., coated) on both sides of the positive electrode current collector 212.

The positive electrode active material 216 may be, or may include, any suitable materials. Suitable positive electrode materials may be selected based on factors such as energy density, interfacial kinetics, electrical conductivity, lithium-ion diffusivity, particle size, particle surface area, density, porosity, and tortuosity, as examples. In one or more embodiments, the positive electrode active material 216 is a material or compound that includes lithium. The lithium included in the positive electrode active material 216 may be doped and undoped during discharging and charging of the battery, respectively. In certain embodiments, the positive electrode active material 216 is, or includes, lithium cobalt oxide (LCO, represented by the formula LiCoO₂). The positive electrode active material 216 may include one or more additional suitable positive electrode active materials, such as lithium-metal oxides (e.g., LiMn₂O₄, Li(NiₓMn_{y}Co_{z})O₂), vanadium oxides, olivines (e.g., LiFePO₄), rechargeable lithium oxides, or manganese dioxide, as examples.

In some embodiments, materials such as binders and conductive additives may be utilized in conjunction with, or within, the layer of the positive electrode active material 216, for example, to bond, or hold, the various positive electrode components together. For example, a layer of coating material including the positive electrode active material (e.g., LCO) may include one or more suitable additives, such as suitable conductive additives and suitable binders. Suitable conductive additives may include, for example, one or more conductive carbon agents, such as graphite, carbon black and/or carbon nanotubes. Suitable binders may include, for example, polyvinylidene fluoride (PVDF) and/or an elastomeric polymer.

The negative electrode 220 includes a negative electrode current collector 222. The negative electrode current collector 222 may be, or include, any suitable material and, in particular, any suitable conductive material, such as copper, a copper alloy, titanium, a titanium alloy, aluminum, or an aluminum alloy. In some embodiments, the negative electrode current collector 222 may be, or include, a foil, such as aluminum foil, an aluminum alloy foil, a titanium foil, a titanium alloy foil, a copper foil, or a copper alloy foil. Negative electrode current collectors that are, or include, a foil may advantageously enable a greater ratio of negative electrode active material to total negative electrode material (e.g., by weight, by volume, etc.). Negative electrode current collectors that are, or include, aluminum/aluminum alloy may be relatively inexpensive, easily formed into a current collector, electrically conductive, readily weldable, corrosion resistant, and generally commercially available. Additionally, negative electrode current collectors that are, or include aluminum/aluminum alloy may advantageously have a relatively low density, which enables a greater ratio of negative electrode active material to total negative electrode material, particularly by weight.

While the positive electrode current collector 212 and the negative electrode current collector 222 are illustrated and described herein as being, or including, a thin foil material, each current collector may have any of a variety of other suitable configurations. Suitable current collector configurations may include, for example, a grid (e.g., a mesh grid), an expanded metal grid, a photochemically etched grid, or a metallized polymer film.

The negative electrode 220 further includes a negative electrode active material 224 disposed (e.g., deposited, coated, etc.) on the negative electrode current collector 222. While FIG. 2A shows the negative electrode active material 224 provided on only one side of the negative electrode current collector 222, a layer of active material similar or identical to the negative electrode active material 224 may be provided (e.g., coated) on both sides of the negative electrode current collector 222, for example, as shown in FIG. 2B.

The negative electrode active material 224 may be, or may include, any suitable materials. Suitable negative electrode materials may be selected based on factors such as energy density, interfacial kinetics, electrical conductivity, lithium-ion diffusivity, particle size, particle surface area, density, porosity, and tortuosity, as examples. In one or more embodiments, the negative electrode active material 224 is, or includes, lithium-titanium-oxide (LTO), represented by the formula Li₄Ti₅O₁₂. The negative electrode active material 224 may include one or more additional suitable negative electrode active materials, such as graphite, lithium, lithium-alloying materials, intermetallic materials (e.g., alloys), and silicon, as examples. In some embodiments, materials such as binders and conductive additives may be utilized in conjunction with, or within, the layer of the negative electrode active material 224, for example, to bond, or hold, the various negative electrode components together.

In some embodiments, as described herein, the positive electrode (e.g., the positive electrode 210 or the positive electrode 20) includes LCO. For example, the positive electrode may include LCO in the positive electrode active material (e.g., the positive electrode active material 216). The positive electrode may include any suitable LCO content. Suitable LCO contents of the positive electrode may be selected based on factors such as desired initial cell energy capacity, desired energy density, desired LCO utilization, desired electronic conductivity, desired ionic conductivity, desired electrode mechanical strength (e.g., for processing, manufacturing, etc.), or desired adhesion strength to the current collector, as examples. While positive electrode LCO content is observed to be positively correlated to improved energy density, higher positive electrode LCO contents are also associated with reduced capacity retention over time (e.g., loss of energy density/energy capacity over the course of charge/discharge cycles). To avoid the loss of capacity over time associated with higher positive electrode LCO contents, LTO/LCO cells constructed outside the preferred ranges described herein are observed to have positive electrodes with lower than preferred LCO contents. For example, LTO/LCO cells constructed outside the preferred ranges described herein are observed to have positive electrodes with LCO contents of less than 90%. Illustrative electrochemical cells described herein may have positive electrodes with LCO content that is 90% or greater, which may advantageously afford improved energy density. Suitable LCO content of the positive electrode may be, for example, between 95% and 97%, or between 90% and 99%. In one embodiment, the positive electrode has an LCO content of approximately 96%. As further examples, suitable positive electrode LCO contents may include 90% or greater, 91% or greater, 92% or greater, 93% or greater, 94% or greater, 95% or greater, 96% or greater, 97% or greater, 98% or greater, or 99% or greater, and/or 99% or less, 98% or less, 97% or less, 96% or less, 95% or less, 94% or less, 93% or less, 92% or less, 91% or less, or 90% or less.

Each of the one or more positive electrodes (e.g., the positive electrode 210 or the positive electrode 20) may have any suitable density. As described herein, relatively dense positive electrodes may afford advantages, such as higher percentages of positive electrode active material in the positive electrode, and higher area specific capacities. Furthermore, while greater densities of the positive electrode may typically be correlated to lower power density (attributable, for example, to reduced ion transfer due to low porosity and high tortuosity or poor electrode properties), positive electrodes with greater densities according to some embodiments described herein may provide improved energy density. Without wishing to be bound by theory, greater LCO densities described herein may provide improved electronic conduction among LCO particles. Positive electrode densities may be affected by factors such as particle size (e.g., particle size of the positive electrode active material), manufacturing (e.g., a compression pressure used in forming the positive electrode), or ratio of current collector to positive electrode active material, as a few examples. Suitable positive electrode densities may be, for example, between 3.7 grams per cubic centimeter (g/cm³) and 4 g/cm³, or between 3 g/cm³ and 4.2 g/cm³. In one embodiment, the positive electrode density is approximately 3.9 g/cm³. As further examples, suitable positive electrode densities may include 3 g/cm³ or greater, 3.2 g/cm³ or greater, 3.5 g/cm³ or greater, 3.7 g/cm³ or greater, 3.9 g/cm³ or greater, 4 g/cm³ or greater, or 4.2 g/cm³ or greater, and/or 4.2 g/cm³ or less, 4 g/cm³ or less, 3.9 g/cm³ or less, 3.7 g/cm³ or less, 3.5 g/cm³ or less, 3.2 g/cm³ or less, or 3 g/cm³ or less.

In some embodiments, the positive electrode active material (e.g., the positive electrode active material 24, 216) includes particles distributed over a range of sizes. A distribution of particle sizes describes the average, minimum, and maximum particle sizes, as well as how the particle sizes are distributed between minimum and maximum particle sizes. In embodiments, distributions are normal. In embodiments, distributions are skewed. In embodiments, distributions are unimodal. In embodiments, distributions are bimodal. In embodiments, distributions are multi-modal (e.g., bi-modal, tri-modal, etc.). Multi-modal particle size distribution may advantageously allow for more dense packing of particles and, thus, higher-density positive electrode active materials. As described herein, higher-density positive electrode active materials may advantageously afford greater energy density. In some embodiments, the positive electrode active material includes particles having a first average particle size (D50) from 10 um to 30 um and includes particles having a second average particle size from 2 um to 8 um. For example, the first average particle size may be from 10 um to 30 um and the second average particle size may be from 2 um to 8 um. As another example, the first average particle size may be 10 um or greater and the second average particle size may be 8 um or less. In some embodiments, the second average particle size may be a percentage of the first average particle size. For example, the second average particle size may be between 5% and 15% or between 1% and 60% the size of the first average particle size. As further examples, the second average particle size may be 60% of the first average particle size or less, 50% of the first average particle size or less, 40% of the first average particle size or less, 30% of the first average particle size or less, 20% of the first average particle size or less, 15% of the first average particle size or less, 10% of the first average particle size or less, or 5% of the first average particle size or less.

Each of the one or more positive electrodes (e.g., the positive electrode 210 or the positive electrode 20) may have any suitable porosity. Porosity may be determined, or measured, using any suitable method or technique, such as Mercury Intrusion Porosimetry or Electromechanical Impedance Spectroscopy, for two examples. Suitable positive electrode porosities may be selected based on desired energy density or material properties such as particle size, particle shape, particle surface area, or desired power density, as examples. Without wishing to be bound by theory, higher porosity generally affords higher power. Suitable positive electrode porosities may be, for example, between 15% and 25%, or between 12% and 35%. In one embodiment, the positive electrode porosity is approximately 18%. As further examples, suitable positive electrode porosities may include 10% or greater, 12% or greater, 15% or greater, 18% or greater, 20% or greater, 22% or greater, 25% or greater, 28% or greater, 30% or greater, or 35% or greater, and/or 35% or less, 30% or less, 28% or less, 25% or less, 22% or less, 20% or less, 18% or less, 15% or less, or 12% or less.

Each of the one or more positive electrodes (e.g., the positive electrode 210) may have any suitable LCO utilization rate. LCO utilization rate may be described as the energy capacity (e.g., in mAh) of the LCO in the positive electrode per unit mass of LCO (e.g., in grams) in the positive electrode. Suitable LCO utilization rates may be selected based on factors such as desired long-term stability, desired positive electrode area specific capacity, or electrolyte material, as examples. As another example, suitable LCO utilization rates may be selected based on desired balance between long-term stability and positive electrode area specific capacity. LCO utilization rate may be affected by factors such as positive electrode thickness and density, cell upper recharge voltage, and negative electrode specific capacity. While relatively high LCO utilization rates may advantageously afford improved cell specific capacity, high LCO utilization rate is also observed to result in reduced long-term stability. To avoid reduced long-term stability associated with high LCO utilization rates, LTO/LCO cells constructed outside the preferred ranges described herein are observed to have lower than preferred LCO utilization rates, lower than preferred long-term stability, or both. For example, in medical device applications, where long-term stability is particularly desirable, LTO/LCO cells constructed outside the preferred ranges described herein are observed to have LCO utilization rates of 120 mAh or less. Suitable LCO utilization rates may be, for example, between 120 mAh/g and 220 mAh/g, between 140 mAh/g and 180 mAh/g, or between 160 mAh/g and 170 mAh/g. In one embodiment, the LCO utilization rate is approximately 165 mAh/g. As further examples, suitable LCO utilization rates may be 120 mAh/g or greater, 130 mAh/g or greater, 140 mAh/g or greater, 150 mAh/g or greater, 160 mAh/g or greater, 165 mAh/g or greater, 170 mAh/g or greater, 180 mAh/g or greater, 190 mAh/g or greater, 200 mAh/g or greater, 210 mAh/g or greater, or 220 mAh/g or greater, and/or 220 mAh/g or less, 210 mAh/g or less, 200 mAh/g or less, 190 mAh/g or less, 180 mAh/g or less, 170 mAh/g or less, 165 mAh/g or less, 160 mAh/g or less, 150 mAh/g or less, 140 mAh/g or less, 130 mAh/g or less, or 120 mAh/g or less.

In some embodiments, as described herein, the negative electrode (e.g., the negative electrode 220 or the negative electrode 30) includes LTO. For example, the negative electrode may include LTO in the negative electrode active material (e.g., the negative electrode active material 224). The negative electrode may include any suitable LTO content, or concentration. Suitable LTO contents of the negative electrode may be selected based on factors such as desired initial cell energy capacity, desired energy density, desired electronic conductivity, desired ionic conductivity, desired electrode mechanical strength (e.g., for processing/manufacturing), or desired adhesion strength to the respective current collector, as examples. While negative electrode LTO content is observed to be positively correlated to improved energy density, higher negative electrode LTO contents are also associated with reduced capacity retention over time (e.g., loss of energy density/energy capacity over the course of charge/discharge cycles). To avoid the loss of capacity over time associated with higher negative electrode LTO contents, LTO/LCO cells constructed outside the preferred ranges described herein are observed to have negative electrodes with lower than preferred LTO contents. For example, LTO/LCO cells constructed outside the preferred ranges described herein are observed to have negative electrodes with LTO contents of less than 90%. Illustrative electrochemical cells described herein may have negative electrodes with LTO content that is 90% or greater, which may advantageously afford improved energy density. Suitable LTO content of the negative electrode may be, for example, between 90% and 96%, or between 85% and 98%. In one embodiment, the negative electrode has an LTO content of approximately 93%. As further examples, suitable negative electrode LTO contents may include 85% or greater, 88% or greater, 90% or greater, 92% or greater, 93% or greater, 95% or greater, 96% or greater, or 98% or greater, and/or 99% or less, 98% or less, 97% or less, 96% or less, 95% or less, 93% or less, 92% or less, 90% or less, 88% or less, or 85% or less.

Each of the one or more negative electrodes (e.g., the negative electrode 220 or the negative electrode 30) may have any suitable porosity. Suitable negative electrode porosities may be selected based on desired energy density or material properties such as particle size, particle shape, particle surface area, or desired power density, as examples. Without wishing to be bound by theory, higher porosity generally affords higher power and lower porosity generally affords greater negative electrode active material loading. Suitable negative electrode porosities may be, for example, between 20% and 50%, or between 31% and 43%. In one embodiment, the negative electrode porosity is approximately 37%. As further examples, suitable negative electrode porosities may include 20% or greater, 25% or greater, 30% or greater, 31% or greater, 35% or greater, 37% or greater, 40% or greater, 43% or greater, 45% or greater, or 50% or greater, and/or 50% or less, 45% or less, 43% or less, 40% or less, 37% or less, 35% or less, 31% or less, 30% or less, 25% or less, or 20% or less.

Each of the one or more negative electrodes (e.g., the negative electrode 220) may have any suitable thickness. Negative electrode thickness may be described as a dimension of the negative electrode along an axis perpendicular (e.g., perpendicular, or nearly perpendicular) to a plane defined by the interface between the negative electrode and a separator. In embodiments including coiled, or wound, electrodes, negative electrode thickness may be described as a dimension of the negative electrode along an axis perpendicular (e.g., perpendicular, or nearly perpendicular) to a plane that is tangent to the interface between the negative electrode and a separator. In some embodiments, such as embodiments with a negative electrode including a negative current collector having a negative electrode active material disposed on both sides of the negative electrode current collector, the negative electrode thickness may include the thickness of the negative electrode active material disposed on both sides of the negative electrode current collector and the thickness of the negative electrode current collector (see, e.g. thickness tₙ in FIG. 2B). Suitable negative electrode thicknesses (e.g., thickness tₙ) may be, for example, between 0.1 millimeters (mm) and 0.2 mm, or between 0.05 mm and 0.5 mm. In one embodiment, the negative electrode thickness is approximately 0.13 mm. As further examples, suitable negative electrode thicknesses may include 0.05 mm or greater, 0.1 mm or greater, 0.13 or greater, 0.15 mm or greater, 0.2 mm or greater, 0.25 mm or greater, 0.3 mm or greater, 0.35 mm or greater, 0.4 mm or greater, 0.45 mm or greater, or 0.5 mm or greater, and/or 0.6 mm or less, 0.5 mm or less, 0.45 mm or less, 0.4 mm or less, 0.35 mm or less, 0.3 mm or less, 0.25 mm or less, 0.2 mm or less, 0.15 mm or less, 0.13 mm or less, 0.1 mm or less, or 0.05 mm or less.

Each of the one or more negative electrodes (e.g., the negative electrode 220) may have any suitable density. As described herein, relatively dense negative electrodes may afford advantages, such as higher percentages of negative electrode active material in the negative electrode, and higher area specific capacities. Negative electrode densities may be affected by factors such as particle size (e.g., particle size of the negative electrode active material), manufacturing (e.g., a compression pressure used in forming the negative electrode), or ratio of current collector to negative electrode active material, as a few examples. Suitable negative electrode densities may be, for example, between 1.5 g/cm³ and 2.5 g/cm³, or between 1.9 g/cm³ and 2.2 g/cm³. In one embodiment, the negative electrode density is approximately 2.1 g/cm³. As further examples, suitable negative electrode densities may include 1.5 g/cm³ or greater, 1.7 g/cm³ or greater, 1.9 g/cm³ or greater, 2 g/cm³ or greater, 2.1 g/cm³ or greater, 2.2 g/cm³ or greater, or 2.5 g/cm³ or greater, and/or 2.5 g/cm³ or less, 2.2 g/cm³ or less, 2.1 g/cm³ or less, 2 g/cm³ or less, 1.9 g/cm³ or less, 1.7 g/cm³ or less, or 1.5 g/cm³ or less.

Negative electrode loading, or deposition, may be described as a mass of negative electrode active material deposited on (e.g., adhered to) a given area of the current collector expressed, for example, in milligrams (mg) of negative electrode active material per square centimeter (cm²) of current collector. While, as described herein, relatively high negative electrode loading may advantageously afford increased percentages of negative electrode active material in the negative electrode and increased negative electrode area specific capacities, high negative electrode loading is also observed to result in reduced rate capability. Furthermore, relatively high negative electrode loading is observed to reduce cell stability, for example, due to limited adhesion between the negative electrode active material and the negative electrode current collector, due to which negative electrode deposited on the current collector may be observed to degrade, for example, during cell assembly or during rolling of a coil electrode assembly. To avoid limited adhesion associated with high negative electrode loading, LTO/LCO cells constructed outside the preferred ranges described herein are observed to have lower than preferred negative electrode loading. For example, LTO/LCO cells constructed outside the preferred ranges described herein are observed to have negative electrode loading of less than 6 mg/cm².

Each of the one or more negative electrodes (e.g., the negative electrode 220) may have any suitable negative electrode active material loading, or deposition. Suitable negative electrode active material loading values may be selected based on factors such as desired negative electrode area specific capacity (described in greater detail below), and may be affected by factors such as negative electrode active material thickness, density, porosity, etc. Suitable negative electrode active material loading values described herein may be afforded, for example, by preparing a slurry of the negative electrode active material and an assistant solvent (e.g., N-Methylpyrrolidone, or NMP). The active material slurry may be coated onto a current collector (e.g., using a knife-over-roll method to apply a consistent layer of active material slurry). The wet layer of active material slurry may be dried, for example, using heat drum drying to remove the assistant solvent. It has been observed that heat drum drying provides advantages compared with other drying methods, such as improved solvent removal, improved mechanical stability of electrode active material layer, and improved adhesion. The dried negative electrode (i.e., the current collector with the dried negative electrode active material coating thereon) may be hot compressed, for example, using a hot calendar roll (e.g., at 75° C.), to afford the desired negative electrode characteristics (e.g., porosity, density, etc.). Hot compression of the negative electrode is observed to reduce, or minimize, stress on the negative electrode during compression, which may advantageously afford a negative electrode with improved mechanical stability and adhesion.

Suitable negative electrode active material loading values may be, for example, between 6 mg/cm² and 60 mg/cm² or between 9 mg/cm² and 15 mg/cm². In one embodiment, the negative electrode active material loading value may be approximately 12 mg/cm². As further example, suitable negative electrode active material loading values may include 6 mg/cm² or greater, 8 mg/cm² or greater, 9 mg/cm² or greater, 10 mg/cm² or greater, 12 mg/cm² or greater, 15 mg/cm² or greater, 20 mg/cm² or greater, 25 mg/cm² or greater, 30 mg/cm² or greater, 40 mg/cm² or greater, 50 mg/cm² or greater, or 60 mg/cm² or greater, and/or 60 mg/cm² or less, 50 mg/cm² or less, 40 mg/cm² or less, 30 mg/cm² or less, 25 mg/cm² or less, 20 mg/cm² or less, 15 mg/cm² or less, 12 mg/cm² or less, 10 mg/cm² or less, 9 mg/cm² or less, 8 mg/cm² or less, or 6 mg/cm² or less.

Each of the one or more negative electrodes (e.g., the negative electrode 220) may have any suitable negative electrode active material thickness. Negative electrode active material thickness may be described as a dimension of the negative electrode active material (e.g., the negative electrode active material 224) along an axis perpendicular (e.g., perpendicular, or nearly perpendicular) to a plane defined by the interface between the respective negative electrode and a separator (see, e.g., thickness tₐ in FIG. 2A). In embodiments including coiled, or wound, electrodes, negative electrode active material thickness may be described as a dimension of the negative electrode active material along an axis perpendicular (e.g., perpendicular, or nearly perpendicular) to a plane that is tangent to the interface between the respective negative electrode and a separator. While negative electrodes with relatively thick negative electrode active materials are observed to afford advantages, such as higher percentages of negative electrode active material in the negative electrode, higher area energy densities, and higher negative electrode active material loading, thicker negative electrode active materials may additionally be associated with reduced mechanical stability. For example, LTO/LCO negative electrodes constructed outside the preferred ranges described herein may include LTO negative electrode active material with micrometer-size spherical secondary particles formed by agglomeration of nanometer-size particles; relatively low adhesion strength between the micrometer-size spherical secondary particles and the current collector may result in reduced cell stability. To avoid reduced mechanical stability associated with high negative electrode active material thickness in LTO/LCO negative electrodes constructed outside the preferred ranges described herein, LTO/LCO negative electrodes constructed outside the preferred ranges described herein are observed to have lower than preferred negative electrode active material thicknesses.

Each of the one or more negative electrodes (e.g., the negative electrode 220) may have any suitable area specific capacity. Area specific capacity of the negative electrode may be defined as the energy density, or specific capacity, of the negative electrode per unit area of negative electrode active material. The area of the negative electrode active material may be defined as the area of the negative electrode active material on a plane defined by an interface between the negative electrode active material and a respective current collector. Negative electrode area specific capacities may be affected by factors such as negative electrode thickness, negative electrode density, and negative electrode loading, each of which may be described as having a positive correlation to negative electrode area specific capacity. As further examples, negative electrode area specific capacity maybe described as positively correlated to negative electrode active material thickness (e.g., deposition thickness of the negative electrode active material on aluminum foil, copper foil, the current collector, etc.), density of the negative electrode, concentration (e.g., weight-percent, volume-percent, etc.) of negative electrode active material in the negative electrode (e.g., percentage of LTO in the negative electrode active material coating layer), and active material utilization (i.e., mass specific capacity, measured, for example, in milliamp-hours per gram). Negative electrode area specific capacity may be determined as the product of negative electrode active material loading (e.g., measured in g/cm²), LTO content (e.g., measured in wt-%), and LTO utilization (e.g., measured in mAh/g). In an example, an illustrative negative electrode having a negative electrode active material loading value of 0.012 g/cm², an LTO content of 93 wt-%, and an LTO utilization of 170 mAh/g is determined to have an area specific capacity of 1.9 mAh/cm².

While, as described herein, relatively high negative electrode area specific capacity may advantageously afford improved cell specific capacity, high negative electrode area specific capacity is also observed to result in reduced stability, for example, due to limited adhesion between the negative electrode active material and the negative electrode current collector. To avoid reduced cell stability associated with high negative electrode area specific capacity, LTO/LCO cells constructed outside the preferred ranges described herein are observed to have lower than preferred negative electrode area specific capacity. For example, LTO/LCO cells constructed outside the preferred ranges described herein are observed to have negative electrode area specific capacities of less than 1.6 mAh/cm². Suitable negative electrode area specific capacities may be, for example, between 1.5 milliamp-hours per square centimeter (mAh/cm²) and 3 mAh/cm², or between 1 mAh/cm² and 15 mAh/cm². In one embodiment, the negative electrode area specific capacity is approximately 2 mAh/cm². As further examples, suitable negative electrode area specific capacities may include 1 mAh/cm² or greater, 1.5 mAh/cm² or greater, 2 mAh/cm² or greater, 3 mAh/cm² or greater, 5 mAh/cm² or greater, 7 mAh/cm² or greater, 10 mAh/cm² or greater, 12 mAh/cm² or greater, or 15 mAh/cm² or greater, and/or 15 mAh/cm² or less, 12 mAh/cm² or less, 10 mAh/cm² or less, 7 mAh/cm² or less, 5 mAh/cm² or less, 3 mAh/cm² or less, 2 mAh/cm² or less, 1.5 mAh/cm² or less, or 1 mAh/cm² or less.

Each of the one or more negative electrodes (e.g., the negative electrode 220) may have any suitable LTO utilization rate. LTO utilization rate may be described as the energy capacity (e.g., in mAh) of the negative electrode per unit mass of LTO (e.g., in grams) in the negative electrode. LTO utilization rates may be affected by factors such as negative electrode thickness and density, negative electrode porosity, cell upper recharge voltage, and positive electrode specific capacity. Relatively high LTO utilization rates may advantageously afford improved cell specific capacity. Suitable LTO utilization rates may be, for example, between 150 mAh/g and 180 mAh/g or between 165 mAh/g and 175 mAh/g. In one embodiment, the LTO utilization rate is approximately 170 mAh/g. It will be understood in light of the present disclosure that any suitable LTO utilization rate may be used, and the disclosure is not limited in this regard. It will be further understood in light of the present disclosure that suitable LCO utilization rates may be selected based on factors, such as those described herein.

Illustrative cells described herein may have any suitable N/P capacity ratio of the negative electrode to the positive electrode. The N/P capacity ratio may be defined as a ratio of the specific capacity, or energy capacity, of the negative electrode to the specific capacity, or energy capacity, of the positive electrode. Suitable N/P capacity ratios may be selected based on factors such as desired cell energy density, desired service life, or desired power capability, as examples. Conventional rechargeable lithium-ion electrochemical cells with graphite or silicon chemistry (i.e., with a negative electrode active material that is graphite or silicon) typically have capacity ratios limited by the energy capacity of the positive electrode (i.e., with N/P capacity ratios greater than 1) due to loss of long-term stability in graphite/silicon cells with N/P capacity ratios of approximately 1 or N/P capacity ratios of less than 1. For example, in graphite/silicon cells with N/P capacity ratios of less than 1, there is a potential for Lithium dendrite formation during charging. Lithium dendrite formation during charging may occur when Lithium ions transferring from the positive electrode cannot find space at the negative electrode and deposit on the surface of the negative electrode.

In contrast, rechargeable lithium-ion electrochemical cells with LTO/LCO chemistry (i.e., with a negative electrode active material that is LTO and a positive electrode active material that is LCO) may have N/P capacity ratios limited by the energy capacity of the negative electrode (i.e., with N/P capacity ratios less than 1) due to long-term stability in LTO/LCO cells with N/P capacity ratios of less than 1, such as N/P capacity ratios between 0.65 and 1. Suitable N/P capacity ratios may be, for example, between 0.7 and 0.85, or between 0.6 and 1. In one embodiment, the N/P capacity ratio is approximately 0.75. As further examples, suitable N/P capacity ratios may include 0.6 or greater, 0.65 or greater, 0.7 or greater, 0.75 or greater, 0.8 or greater, 0.85 or greater, 0.9 or greater, 0.95 or greater, 0.97 or greater, or 1 or greater.

Electrochemical cells constructed according to illustrative embodiments and preferred ranges described herein may be designed with upper recharge voltages greater than LTO/LCO cells constructed outside the preferred ranges described herein. The upper recharge voltage of a cell (e.g., the electrochemical cell 200) may be described as the voltage across a positive electrode (e.g., the positive electrode 210) and a negative electrode (e.g., the negative electrode 220) when the cell is fully charged. While greater upper recharge voltages may generally be described as contributing to increased LCO utilization, greater upper recharge voltages may also be correlated to faster degradation and, thus, reduced long-term cell stability. To avoid increased degradation and reduced long-term cell stability associated with high upper recharge voltages and high LCO utilization in LTO/LCO cells constructed outside the preferred ranges described herein, LTO/LCO cells constructed outside the preferred ranges described herein are observed to have lower than preferred upper recharge voltages, operating voltages, and LCO electrode utilization. For example, LTO/LCO cells constructed outside the preferred ranges described herein are observed to have upper recharge voltages limited to 2.8 volts (V) or less. Illustrative electrochemical cells described herein may be designed with upper recharge voltages of 2.8 V or greater, which may advantageously afford improved recharge rates, increased average cell voltage during use, improved energy, and improved specific capacity/energy density. In some embodiments, for example, the electrochemical cell has an upper recharge voltage of between 2.8 V and 3 V, or between 2.6 V and 3.4 V. In one embodiment, the upper recharge voltage is approximately 2.8 V. As further examples, the electrochemical cell may have upper recharge voltages of 2.6 V or greater, 2.8 V or greater, 2.9 V or greater, 3 V or greater, 3.1 V or greater, 3.2 V or greater, 3.3 V or greater, or 3.4 V or greater, and/or 3.4 V or less, 3.3 V or less, 3.2 V or less, 3.1 V or less, 3 V or less, 2.9 V or less, or 2.8 V or less.

Illustrative electrochemical cells described herein may provide improved charge and discharge rates. In some embodiments, for example, the electrochemical cell can be discharged at a discharge rate of between C/10 and C/0.33, or between C/10,000 and C/0.1. In one embodiment, the electrochemical cell can be discharged at a discharge rate of approximately C/1. As further examples, the electrochemical cell may have discharge rates of C/0.1 or greater, C/0.33 or greater, C/0.5 or greater, C/1 or greater, C/10 or greater, C/100 or greater, C/1,000 or greater, or C/10,000 or greater, and/or C/10,000 or less, C/1,000 or less, C/100 or less, C/10 or less, C/1 or less, C/0.5 or less, C/0.33 or less, or C/0.1 or less. Similarly, illustrative electrochemical cells described herein may provide improved charge rates. In some embodiments, for example, the electrochemical cell can be charged at a charge rate of between C/1 and C/0.25, or between C/100 and C/0.1. In one embodiment, the electrochemical cell can be charged a charge rate of approximately C/0.5. As further examples, the electrochemical cell may have charge rates of C/0.1 or greater, C/0.25 or greater, C/0.5 or greater, C/1 or greater, C/10 or greater, C/30 or greater, C/50 or greater, or C/100 or greater, and/or C/100 or less, C/50 or less, C/30 or less, C/10 or less, C/1 or less, C/0.5 or less, C/0.25 or less, or C/0.1 or less.

In one or more embodiments, as described herein, the electrochemical cell may include one or more separators (e.g., the separator 250 of FIG. 2), which may each be between electrodes of opposite polarities. In other words, each of the one or more separators may be disposed or sandwiched between electrodes of opposite polarities, such as between a negative electrode (e.g., the negative electrode 220) and a positive electrode (e.g., the positive electrode 210) and may further be in intimate contact with said electrodes. The one or more separators may each be porous, microporous, perforated, or may include holes for electrolyte material to penetrate the separator. Accordingly, each of the one or more separators may facilitate ion transfer within the electrochemical cell because an electrolyte material provides a medium for ion transfer.

The one or more separators may each be made of any suitable material or combination of materials. Suitable separator materials may be selected based on porosity, tortuosity, or mechanical strength, as just a few examples. Suitable separator materials may include, for example polypropylene, polyethylene, Polytetrafluoroethylene (PTFE), cellophane, nylon, polyolefin, microporous membrane, or multilayer microporous membrane (e.g., CELGARD® 2320 Trilayer Microporous Membrane). It will be understood in light of the present disclosure that any suitable separator materials may be used, and the disclosure is not limited in this regard. It will be further understood in light of the present disclosure that suitable separator materials may vary depending on factors, including those described herein.

In one or more embodiments, as described herein, the electrochemical cell (e.g., the electrochemical cell 200) includes an electrolyte material. The electrolyte material may generally fill at least a portion of any spaces inside the housing not filled by the other components of the electrochemical cell. The electrochemical cell may include a volume not filled by electrolyte material (that is, a void). The void may be useful, for example, to avoid overpressure of the enclosure. The electrolyte facilitates ion transfer between opposite-polarity electrodes, such as between a negative electrode (e.g., the negative electrode 220) and a positive electrode (e.g., the positive electrode 210). The electrolyte material may have an electrical potential. The electrolyte material may include any suitable material and may be one or more of, for example, a liquid, a gel, a solid, or a paste. The material composition of the electrolyte material may include, for example, lithium salt or another suitable electrolyte. The electrolyte material may include a non-aqueous solution in which a lithium salt (for example, lithium hexafluorophosphate salt) is dissolved in an organic carbonate solvent (such as, for example, mixtures including one or more of ethylene carbonate, dimethyl carbonate, propylene carbonate, diethyl carbonate, or ethyl methyl carbonate).

In some embodiments, the electrolyte material includes a high-voltage electrolyte additive, such as vinylene carbonate (VC). Electrochemical cells constructed according to illustrative embodiments and preferred ranges described herein including VC as a high-voltage electrolyte additive may be designed with upper recharge voltages greater than LTO/LCO cells constructed outside the preferred ranges described herein. Additionally or alternatively, electrochemical cells constructed according to illustrative embodiments and preferred ranges described herein including VC as a high-voltage electrolyte additive may have improved long-term cell stability compared with LTO/LCO cells constructed outside the preferred ranges described herein. As described herein, relatively greater upper recharge voltages may generally be described as contributing to reduced long-term cell stability. To mitigate or avoid reduced long-term cell stability associated with high upper recharge voltages in LTO/LCO cells constructed outside the preferred ranges described herein, LTO/LCO cells constructed outside the preferred ranges described herein are observed to have electrolyte materials with greater than preferred concentrations of high-voltage electrolyte additives and, particularly, higher than preferred concentrations of high-voltage electrolyte additives other than VC, such as commercial high-voltage electrolyte additives. For example, to mitigate or avoid reduced long-term cell stability associated with high upper recharge voltages in LTO/LCO cells constructed outside the preferred ranges described herein, LTO/LCO cells constructed outside the preferred ranges described herein and with high upper recharge voltages are observed to have electrolyte materials with high-voltage electrolyte additive concentrations of 2% or greater and, particularly, electrolyte materials with high-voltage electrolyte additives other than VC in concentrations of 2% or greater. Additionally, LTO/LCO cells constructed outside the preferred ranges described herein and including high-voltage stable electrolyte materials outside the preferred ranges described herein are observed to have lower than preferred long-term cell stability.

In one or more embodiments, the electrolyte material includes at least one high-voltage additive. In embodiments, the at least one high-voltage additive includes vinylene VC. In some embodiments, the at least one high-voltage additive includes VC and further includes other high-voltage additives (i.e., high-voltage additives other than VC). In embodiments including other high-voltage additives, the electrolyte material may have a VC content that is greater than the content of other high-voltage additives. In some embodiments including other high-voltage additives, the electrolyte material may have a content of other high-voltage additives that is less than 2%, less than 1.5%, less than 1%, or less than 0.5%. In some embodiments, the electrolyte material includes VC as a high-voltage additive and includes substantially no other high-voltage additives (i.e., substantially no high-voltage additives other than VC, e.g., trace amounts of other high-voltage additives or less than trace amounts of other high-voltage additives). In embodiments, the electrolyte material includes VC as a high-voltage additive and includes no other high-voltage additives (i.e., no high-voltage additives other than VC).

As described herein, VC in the electrolyte material may afford advantages, such as suppressing gas generation during formation/manufacturing, for example. As another example, VC in the electrolyte material may advantageously improve long-term stability of the electrochemical cell, such as by improving active material stability and reducing cell resistance growth. Increased long-term cell stability may advantageously enable improved upper recharge voltages, LCO utilization, LTO utilization, and energy densities. The electrolyte material may include any suitable concentration of VC, or any suitable VC content. Suitable concentrations of VC may be selected based on factors such as material compatibility (e.g., with other constituents of the electrolyte material, with the electrode materials, with the separator, etc.), desired positive electrode stability, desired electrolyte stability at high voltages, desired stability of the surface of the positive electrode, or desired stability of the surface of the negative electrode, as examples. Suitable concentrations of VC may be, for example, between 0.5 percent by weight (weight-percent, or wt-%) and 5 wt-%. In one embodiment, the electrolyte material has a VC concentration of approximately 3 wt-%. As further examples, suitable VC concentrations may include 0.5 wt-% or greater, 1 wt-% or greater, 1.5 wt-% or greater, 2 wt-% or greater, 2.5 wt-% or greater, 3 wt-% or greater, 3.5 wt-% or greater, 4 wt-% or greater, 4.5 wt-% or greater, or 5 wt-% or greater, and/or 5 wt-% or less, wt-% or less, 4.5 wt-% or less, 4 wt-% or less, 3.5 wt-% or less, 3 wt-% or less, 2.5 wt-% or less, 2 wt-% or less, 1.5 wt-% or less, 1 wt-% or less, or 0.5 wt-% or less.

The illustrative electrochemical cells described herein may have any suitable electrolyte content. In some embodiments, electrolyte content may be described as a mass of the electrolyte material in the electrochemical cell per energy capacity of the electrochemical cell. Suitable electrolyte contents may be, for example, between 5 grams per Amp-hour (g/Ah) and 7 g/Ah, or between 2 g/Ah and 10 g/Ah. In one embodiment, the electrolyte content is approximately 6 g/Ah. As further examples, suitable electrolyte contents may include 2 g/Ah or greater, 3 g/Ah or greater, 4 g/Ah or greater, 5 g/Ah or greater, 6 g/Ah or greater, 7 g/Ah or greater, 8 g/Ah or greater, 9 g/Ah or greater, or 10 g/Ah or greater.

In one or more embodiments, as described herein, the electrochemical cell may be at least partially disposed in a housing. Although not explicitly shown in the figures, the housing may generally enclose the components of the electrochemical cell and contain the electrolyte material within the housing. At least portions of at least some components may not be enclosed by the housing. Portions of each of the one or more current collectors may not be enclosed by the housing, as an example.

The housing may include any suitable material or combination of materials. Suitable housing materials may include aluminum, titanium, stainless steel, nickel, and nickel coated ferrous steels, as examples. In one or more embodiments, the housing may include a polymeric material. It will be understood in light of the present disclosure that any suitable housing materials may be used, and the disclosure is not limited in this regard. It will be further understood in light of the present disclosure that suitable housing materials may vary depending on factors, including those described herein.

In some embodiments, the electrochemical cell may have a “case neutral” design. In other words, the housing may float according to the electrical potential of the electrochemical cell. To achieve a case neutral design, the electrochemical cell may include one or more positive electrode current collectors (e.g., the positive electrode current collector 212) and one or more negative electrode current collectors (e.g., the negative electrode current collector 222) that extend through the housing while being insulated from the housing by a feedthrough insulator.

In various embodiments, the electrochemical cell may include various insulators (not shown in the figures) to insulate the conductive components (such as the housing, the one or more current collectors, the negative electrodes, and the positive electrodes, for a few examples) from one another. The insulators may be made of any suitable material or combination of materials. Suitable insulator materials may include, for example, polytetrafluoroethylene (PTFE), polysulfone, glass, and ceramic materials (such as alumina). It will be understood in light of the present disclosure that any suitable insulator materials may be used, and the disclosure is not limited in this regard. It will be further understood in light of the present disclosure that suitable insulator materials may vary depending on factors, including those described herein.

In one or more embodiments, the electrochemical cell may include various electrical connections, such as between conductive components. Such electrical connections may be made by intimate contact between two or more conducting materials. Additionally or alternatively, such electrical connections may be made by welding two or more conducting materials together (e.g., by resistance welding or laser welding). Where conducting materials have at least slightly incompatible metallurgical characteristics (such as in a connection between titanium and copper), a weld interposer (e.g., a vanadium weld interposer) may be used to manage weld stability and strength.

According to an exemplary embodiment, lithium-ion batteries such as those described above may be used in conjunction with medical devices such as medical devices that may be implanted in the human body (referred to as “implantable medical devices” or “IMD's”). Some examples of implantable medical devices that may include a battery as described herein include cardiac defibrillator, cardiac pacemakers, cardioverters, cardiac contractility modulators, drug infusion devices, diagnostic recorders, hearing aids, sensors, telemetry devices, cochlear implants, neurological stimulation devices, and the like. Such devices may be used for monitoring or alleviating the adverse effects of various health ailments. According to still other embodiments, non-implantable medical devices or other types of devices may utilize batteries as are shown and described in this disclosure.

It is also contemplated that the medical devices described herein may be charged or recharged when the medical device is implanted within a patient. That is, according to an example embodiment, there is no need to disconnect or remove the medical device from the patient in order to charge or recharge the medical device. For example, transcutaneous energy transfer (TET) may be used, in which magnetic induction is used to deliver energy from outside the body to the implanted battery, without the need to make direct physical contact to the implanted battery, and without the need for any portion of the implant to protrude from the patient's skin.

It should be understood that while the present disclosure describes the use of lithium-ion batteries with a variety of medical devices, such batteries may be used in a variety of other applications, including computers (e.g., laptop computers), phones (e.g., cellular, mobile, or cordless phones), automobiles, and any other device or application for which it may be advantageous to provide power in the form of a battery as described herein.

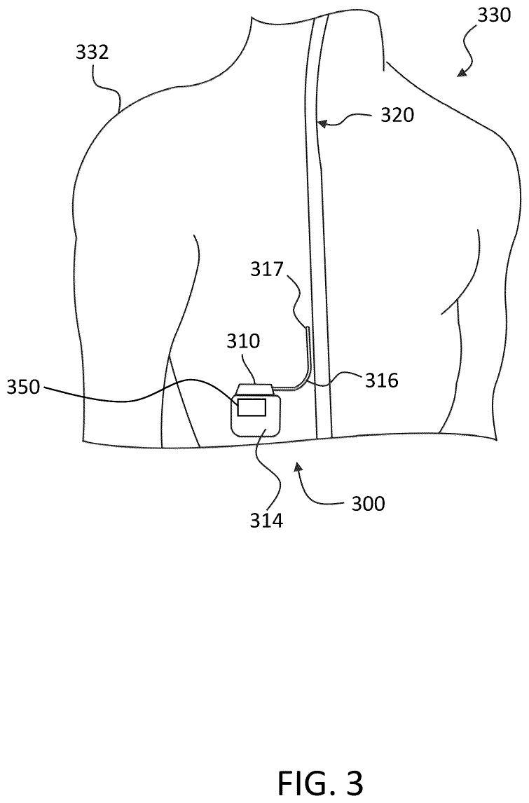

Referring now to FIG. 3, an example of a system 300 (e.g., an implantable medical device) implanted within a body or torso 332 of a patient 330 is shown. The system 300 includes a device 310 in the form of an implantable medical device that for purposes of illustration is shown as a neurostimulator configured to provide a therapeutic treatment for the patient 330.

The device 310 includes a container or housing 314 that is hermetically sealed and biologically inert according to an exemplary embodiment. The container may be made of a conductive material. One or more leads 316 electrically connect the device 310 to a target neurological structures, such as nerve fibers near the patient's spinal column 320. Electrodes 317 are provided to sense electrical activity (e.g., nerve activity) and/or provide electrical stimulation to the neurological structure. At least a portion of the leads 316 (e.g., an end portion of the leads shown as exposed electrodes 317) may be provided adjacent or in contact with the target neurological structure.

The device 310 includes a battery 350 provided therein to provide power for the device 310. The battery 350 is a battery as described herein.

### Illustrative Aspects

Aspect 1 is a rechargeable lithium-ion cell comprising:
a negative electrode comprising a negative electrode active material comprising Li₄Ti₅O₁₂ (LTO), the negative electrode having: a negative electrode area specific capacity of 2 milliAmp-hours per square centimeter (mAh/cm²), between 1.5 mAh/cm² and 3 mAh/cm², or between 1 mAh/cm² and 15 mAh/cm²;
a positive electrode comprising a positive electrode active material comprising LiCoO₂ (LCO), the positive electrode having: a density of 3.9 grams per cubic centimeter (g/cm³), between 3.7 g/cm³ and 4 g/cm³, or between 3 g/cm³ and 4.2 g/cm³;
a separator between the negative electrode and the positive electrode; and
an electrolyte material comprising from 0.5 to 5 percent by weight vinylene carbonate;
wherein the cell has a cell upper recharge voltage of 2.8 volts (V), between 2.6 V and 3 V, or between 2.6 V and 3.4 V.

Aspect 2 is a rechargeable lithium-ion cell comprising:
a negative electrode comprising a negative electrode active material comprising LTO, the negative electrode having: a negative electrode active material loading value of 10 milligrams per square centimeter (mg/cm²) or greater, between 6 mg/cm² and 60 mg/cm², or between 9 mg/cm² and 15 g/cm²;
a positive electrode comprising a positive electrode active material comprising LCO, the positive electrode having: a LCO utilization rate of 130 milliAmp-hours per gram (mAh/g) or greater, between 120 mAh/g and 220 mAh/g, or between 140 mAh/g and 180 mAh/g;
a separator between the negative electrode and the positive electrode; and
an electrolyte material comprising from 0.5 to 5 percent by weight vinylene carbonate as a high-voltage additive;
wherein the cell has a cell upper recharge voltage of 2.8 volts (V), between 2.6 V and 3 V, or between 2.6 V and 3.4 V.

Aspect 3 is a rechargeable lithium-ion cell comprising:
a negative electrode comprising a negative electrode active material comprising LTO, the negative electrode having: a negative electrode area specific capacity of 2 milliAmp-hours per square centimeter (mAh/cm²), between 1.5 mAh/cm² and 3 mAh/cm², or between 1 mAh/cm² and 15 mAh/cm²;
a positive electrode comprising a positive electrode active material comprising LCO, the positive electrode having: a LCO utilization rate of 130 milliAmp-hours per gram (mAh/g) or greater, between 120 mAh/g and 220 mAh/g, between 140 mAh/g and 180 mAh/g, or between 160 mAh/g and 170 mAh/g;
a separator between the negative electrode and the positive electrode; and
an electrolyte material comprising from 0.5 to 5 percent by weight vinylene carbonate as a high-voltage additive;
wherein the cell has a cell upper recharge voltage of 2.8 volts (V), between 2.6 V and 3 V, or between 2.6 V and 3.4 V.

Aspect 4 is the cell of any one of aspects 1-3, wherein the LCO of the positive electrode active material comprises particles having a multi-modal size distribution.

Aspect 5 is the cell of aspect 4, wherein the LCO of the positive electrode active material comprises particles having a first average particle size and particles having a second average particle size; and wherein the first average particle size is between 10 um and 30 um and the second average particle size is between 2 um and 8 um.

Aspect 6 is the cell of aspect 4, wherein the LCO of the positive electrode active material comprises particles having a first average particle size and particles having a second average particle size; and wherein the second average particle size is between 5% and 15% the size of the first average particle size.

Aspect 7 is the cell of any one of aspects 1-6, wherein the positive electrode further comprises a binder comprising polyvinylidene fluoride (PVDF).

Aspect 8 is the cell of any one of aspects 1-7, wherein the positive electrode further comprises a carbon conductive agent comprising carbon black, graphite, or a combination thereof.

Aspect 9 is the cell of any one of aspects 1-8, wherein the positive electrode has a porosity of 18%, between 15% and 25%, or between 12% and 35%.

Aspect 10 is the cell of any one of aspects 1-9, wherein the positive electrode has an LCO utilization of 130 milliAmp-hours per gram (mAh/g) or greater, between 120 mAh/g and 220 mAh/g, or between 140 mAh/g and 180 mAh/g.

Aspect 11 is the cell of any one of aspects 1-10, wherein the negative electrode has a thickness of 0.13 millimeters (mm), between 0.1 mm and 0.2 mm, or between 0.05 mm and 0.5 mm.

Aspect 12 is the cell of any one of aspects 1-11, wherein the negative electrode has an active material loading value of 10 mg/cm² or greater, between 6 mg/cm² and 60 mg/cm², or between 9 mg/cm² and 15 mg/cm².

Aspect 13 is the cell of any one of aspects 1-12, wherein the negative electrode has a porosity of 37%, between 20% and 50%, or between 31% and 43%.

Aspect 14 is the cell of any one of aspects 1-13, wherein the negative electrode has an LTO utilization of 170 mAh/g, between 150 mAh/g and 180 mAh/g, or between 165 mAh/g and 175 mAh/g.

Aspect 15 is the cell of any one of aspects 1-14, wherein the negative electrode comprises an LTO content of 93%, between 90% and 96%, or between 85% and 98%.

Aspect 16 is the cell of any one of aspects 1-15, wherein the positive electrode comprises an LCO content of 96%, between 95% and 97%, or between 90% and 99%.

Aspect 17 is the cell of any one of aspects 1-16, wherein the negative electrode has a negative electrode area specific capacity of 2 mAh/cm², between 1.5 mAh/cm² and 3 mAh/cm², or between 1 mAh/cm² and 15 mAh/cm².

Aspect 18 is the cell of any one of aspects 1-17, wherein the positive electrode has a LCO utilization rate of 130 mAh/g or greater, between 120 mAh/g and 220 mAh/g, between 140 mAh/g and 180 mAh/g, or between 160 mAh/g and 170 mAh/g.

Aspect 19 is the cell of any one of aspects 1-18, wherein the positive electrode has a density of 3.9 g/cm³, between 3.7 g/cm³ and 4 g/cm³, or between 3 g/cm³ and 4.2 g/cm³.

Aspect 20 is the cell of any one of aspects 1-19, wherein the positive electrode has a positive electrode area specific capacity, and wherein a N/P capacity ratio of the negative electrode area specific capacity to the positive electrode area specific capacity is 0.75:1, between 0.65:1 and 0.85:1, or between 0.5:1 and 1:1.

Aspect 21 is the cell of any one of aspects 1-20, wherein the cell comprises an electrolyte content of 6 grams per Amp-hour (g/Ah), between 5 g/Ah and 7 g/Ah, or between 2 g/Ah and 10 g/Ah.

Aspect 22 is the cell of any one of aspects 1-21, wherein the cell can be charged at a charge rate of C/0.5, between C/1 and C/0.25, or between C/100 and C/0.1.

Aspect 23 is an implantable medical device comprising the cell of any one of aspects 1-22.

It should further be noted that, as used in this specification and the appended claims, reference to numbers of electrodes is merely for the purpose of distinguishing between electrodes and does not necessarily limit the number of electrodes.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

### EXAMPLES

### Sample Preparation

TABLE 1
Parameters used in modelling the Samples

| | Sample 1 | | Sample 2 | |
|---|---|---|---|---|
| Form Factor | Stack-plate cell | | Coiled cell | |
| Negative electrode material | LTO | | LTO | |
| Negative electrode area | 2.1-2.4 | mAh/cm² | 2.1-2.4 | mAh/cm² |
| specific capacity | | | | |
| Positive electrode material | LCO | | LCO | |
| Positive electrode LCO | 115-145 | mAh/g | 130-205 | mAh/g |
| utilization | | | | |
| Electrolyte to cell capacity | 5.3 | g/Ah | 10 | g/Ah |
| ratio | | | | |

Sample electrochemical cells were prepared according to illustrative embodiments and preferred ranges described herein. The samples were hermetic cells with titanium cases prepared according to the parameters described in Table 1. The LTO and LCO electrodes were each prepared using a slurry coating method. For the positive electrodes, LCO, conductive carbon agent (carbon black and graphite), PVDF binder, and N-methylpyrrolidone (NMP, as an assistant solvent) were mixed into a flowable slurry. For the negative electrodes, LTO, conductive carbon agent (carbon black and graphite), PVDF binder, and NMP (as an assistant solvent) were mixed into a flowable slurry. The respective slurries were coated on aluminum sheets (i.e., aluminum current collectors) using a knife-over-roll method, providing wet electrode coatings on the respective aluminum sheets. The wet electrode coatings were then dried in a heat drum drying chamber, removing the NMP. Each dried electrode coating on the aluminum sheet was compressed using a calendar roll to achieve the respectively desired porosity. Negative electrodes and positive electrodes were cut from the respective sheets and assembled into respective electrode configurations (i.e., stack for Sample 1 and coiled for Sample 2). The electrode configurations were assembled into respective titanium cases, providing dry cell assemblies. Electrolyte material was added to each dry cell assembly via a fill port to a desired electrolyte level, and then the fill port was sealed. Each cell underwent a charge-discharge cycle to activate cell chemistry before further electrochemical testing.

### Sample Analysis—LCO Utilization

As described herein, while LCO utilization is positively correlated with initial cell capacity, or energy density, high LCO utilization typically results in reduced capacity retention over time. For 90% capacity retention after 15 years of daily rate cycling at 37° C. (i.e., after 15 years of 2C charge and C/24 discharge cycles between 2.8 V and 1.8 V at ambient body temperature, the energy capacity of a cell is 90% of the cell's initial energy capacity), LTO/LCO cells constructed outside the preferred ranges described herein are observed to typically have LCO utilization of 130 mAh/g or less to achieve 10 years of service life or greater. Additionally, while some LTO/LCO cells constructed outside the preferred ranges described herein may have relatively high LCO utilization (e.g., 130 mAh/g or greater), such cells are observed to have relatively short service lives, such by having less than 90% rate retention in 15 years or less, 10 years or less, 5 years or less, 3 years or less, or 2 years or less.

In accelerated daily rate cycling tests, illustrative electrochemical cells according to embodiments disclosed herein underwent daily rate cycling at 75° C., which provides an acceleration factor of 52.6 compared to daily rate cycling at 37° C. (i.e., ambient body temperature). In other words, 105 days of accelerated daily rate cycling at 75° C. is equivalent to 15 years of daily rate cycling at 37° C.

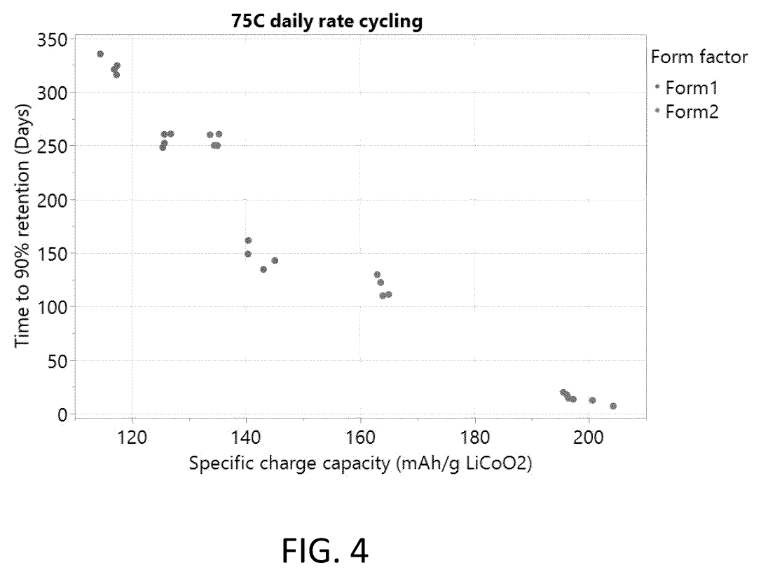

A graphical representation of the results of 75° C. accelerated daily rate cycling tests for illustrative electrochemical cells with various LCO utilization value between 120 mAh/g and 200 mAh/g, according to embodiments described herein, are shown in FIG. 4. Illustrative electrochemical cells were prepared according to embodiments described herein with various LCO utilization value between 120 mAh/g and 200 mAh/g, electrolyte material including 2% VC, electrolyte-cell capacity ratio of 5.3 g/Ah, and LTO area specific capacity of 2.1-2.4 mAh/cm². In addition to various LCO utilization values, illustrative electrochemical cells were tested with stack-plate cell form factors (Form1) and coiled cell form factors (Form2). Each illustrative cell underwent 75° C. accelerated daily rate cycling until the energy capacity of the cell reached 90% capacity retention. The horizontal axis represents the LCO utilization of each illustrative cell (in mAh/g LiCoO₂). The vertical axis represents the number of days each illustrative cell underwent 75° C. accelerated daily rate cycling before the cell reached 90% capacity retention. As shown in FIG. 4, illustrative electrochemical cells with LCO utilization of 165 mAh/g or less afford 90% capacity retention after 105 days of 75° C. accelerated daily rate cycling. In other words, illustrative electrochemical cells according to embodiments described herein having LCO utilization of 165 mAh/g or less would have 90% capacity retention after 15 years of daily rate cycling at 37° C.

### Example Analysis—LTO Area Specific Capacity

As described herein, while area specific capacity of LTO negative electrodes (i.e., negative electrodes having a negative electrode active material including LTO) is positively correlated to cell energy density, high area specific capacity typically results in reduced rate capability. Furthermore, as discussed herein, to avoid reduced cell stability associated with high negative electrode area specific capacity, LTO/LCO cells constructed outside the preferred ranges described herein are observed to have lower than preferred negative electrode area specific capacity. For example, LTO/LCO cells constructed outside the preferred ranges described herein are observed to have negative electrode area specific capacities of less than 1.6 mAh/cm².

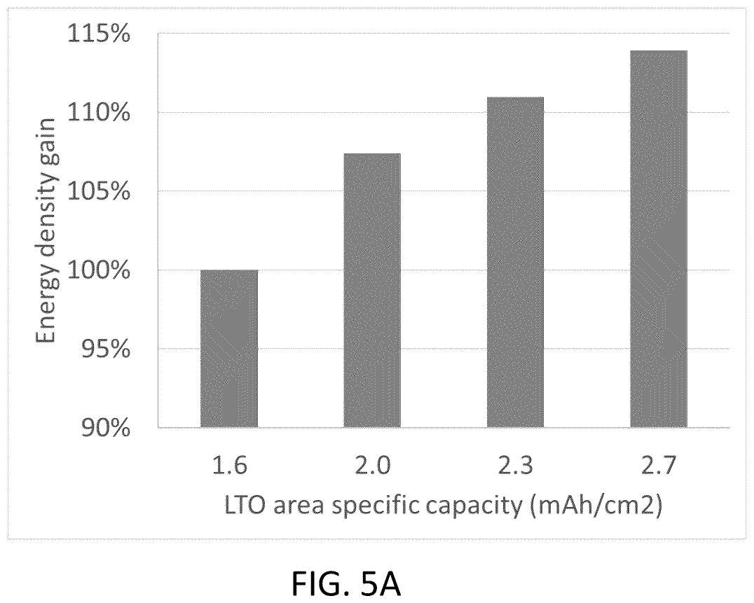

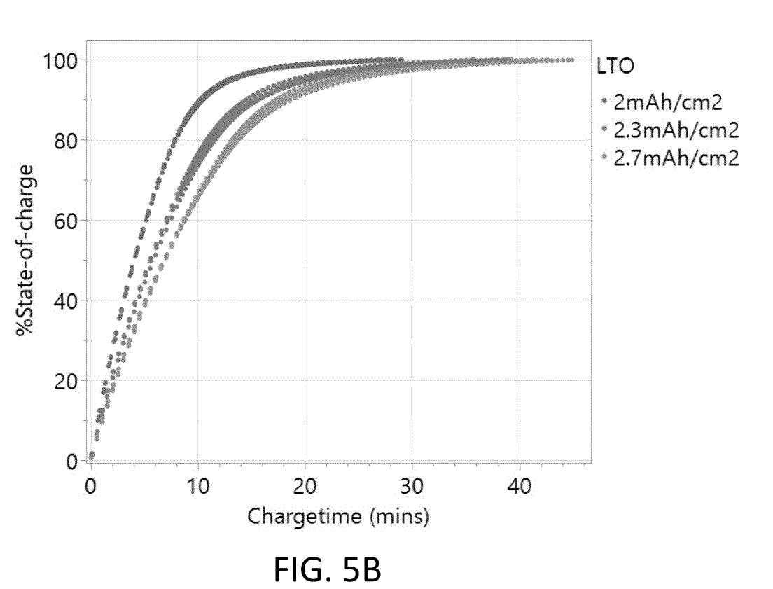

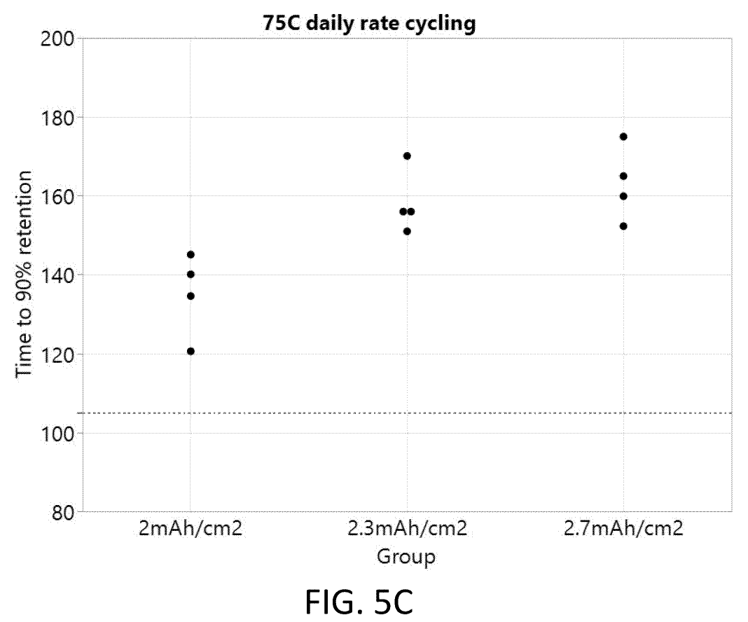

A graphical representation comparing the energy densities of a comparative electrochemical cell with typical LTO area specific capacity (1.6 mAh/cm²) to illustrative electrochemical cells according to embodiments disclosed herein with relatively higher LTO area specific capacities is shown in FIG. 5A. The illustrative electrochemical cells were prepared according to embodiments described herein with various, relatively higher, LTO area specific capacities and with constant internal space, separator thickness, and current collector thickness. The vertical axis represents the calculated energy density of each cell relative to the energy density of the comparative cell. Energy densities for illustrative electrochemical cells having coiled electrode design and various LTO area specific capacities were modelled with other cell parameters constant, such as electrode density, electrolyte amount (in grams per Amp-hour), characteristics of inert components (e.g., separator, current collector, inner connections, etc.), available internal volume of the cell, N/P ratio, and LCO utilization. The horizontal axis represents the LTO area specific capacity of each cell (in mAh/cm²). As shown in FIG. 5A, higher LTO area specific capacity increased energy density by up to 14%.

A graphical representation of recharge rates for the illustrative cells with high LTO area specific capacity is shown in FIG. 5B. The vertical axis represents the state-of-charge (as a percentage, where 100% represents fully charged). The horizontal axis represents the time elapsed during charging (in minutes). As shown in FIG. 5B, each illustrative cell can be recharged to 95% or greater within 20 minutes.

A graphical representation of the results of 75° C. accelerated daily rate cycling tests (as described above) for the illustrative electrochemical cells with high LTO area specific capacity is shown in FIG. 5C. The horizontal axis represents the LTO area specific capacity of each illustrative cell. The vertical axis represents the number of days each illustrative cell underwent 75° C. accelerated daily rate cycling before the cell reached 90% capacity retention. As shown in FIG. 5C, each illustrative cell reached 90% capacity after 105 days or more of 75° C. accelerated daily rate cycling. In other words, the illustrative electrochemical cells with high LTO area specific capacities would have at least 90% capacity retention after 15 years of daily rate cycling at 37° C.

### Example Analysis—Electrolyte Material With Vinylene Carbonate (VC) Additive

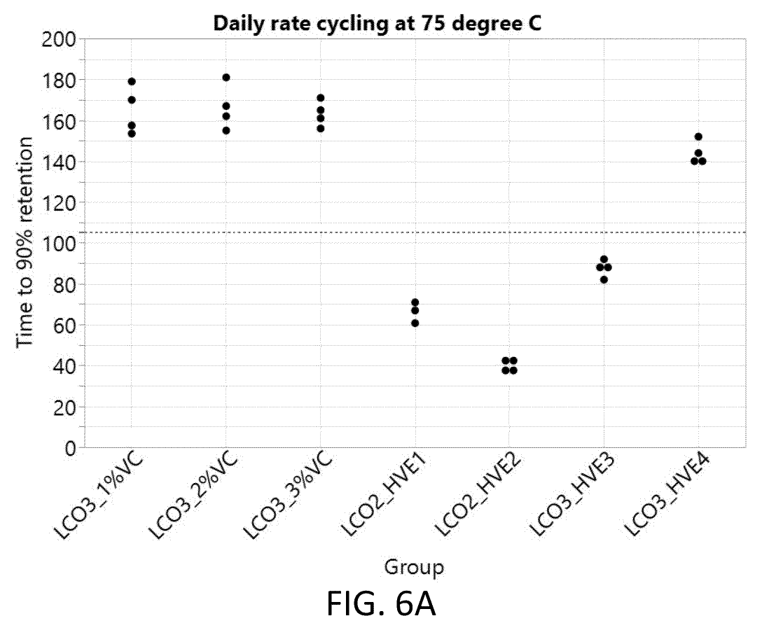

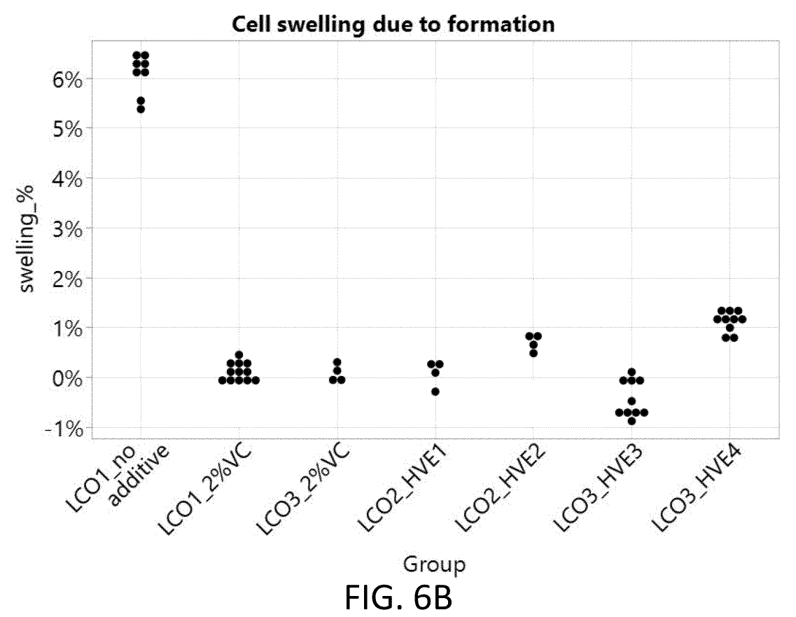

As discussed herein, electrolyte materials including high-voltage additives, such as VC, may be useful, for example, to improve long-term stability of cells (e.g., capacity retention) and to suppress (e.g., prevent) gas generation during manufacturing or formation. Graphical representations of the results of studies comparing cell stability and gas formation of illustrative electrochemical cells with electrolyte material including VC and comparative cells are shown in FIGS. 6A and 6B. Each electrochemical cell was formed as a hermetically sealed prismatic cell using used LCO/LTO electrode chemistry, LTO area specific capacity of 2 mAh/cm², LCO utilization of 165 mAh/g, and N/P ratio of 0.75. Each cell included a base electrolyte material composition of 1.1M LiPF₆/EC:EMC, to which respective additives were added.

A graphical representation of the results of 75° C. accelerated daily rate cycling tests (as described above) for illustrative electrochemical cells with electrolyte material including VC is shown in FIG. 6A. The horizontal axis represents the electrolyte additive composition of each cell, including illustrative electrochemical cells with 1% VC (LCO3_1% VC), 2% VC (LCO3_2% VC), and 3% VC (LCO3_3% VC). Comparative electrochemical cells underwent the 75° C. accelerated daily rate cycling test, including cells with four comparative commercial high-voltage electrolyte materials, HVE1 (LCO2_HVE1), HVE2 (LCO2_HVE2), HVE3 (LCO3_HVE3), and HVE4 (LCO3_HVE4). The vertical axis represents the number of days each cell underwent 75° C. accelerated daily rate cycling before the cell reached 90% capacity retention. As shown in FIG. 6A, each illustrative cell (1% VC, 2% VC, and 3% VC) underwent at least 105 days (and, in fact, more than 140 days) of 75° C. accelerated daily rate cycling before the cell reached 90% capacity retention. In other words, the illustrative electrochemical cells with electrolyte material including VC would have at least 90% capacity retention after 15 years of daily rate cycling at 37° C. It has been observed that the illustrative electrochemical cells with VC electrolyte materials unexpectedly outperform the cells with comparative commercial high-voltage electrolyte materials in 75° C. accelerated daily rate cycling tests. More specifically, one cell with comparative electrolyte material (LCO3_HVE4) underwent at least 105 days of 75° C. accelerated daily rate cycling before the reaching 90% capacity retention and three cells with comparative electrolyte materials (LCO2_HVE1, LCO2_HVE2, and LCO3_HVE3) were each observed to undergo fewer than 105 days of 75° C. accelerated daily rate cycling before the cell reached 90% capacity retention.

A graphical representation of swelling measured after formation of illustrative electrochemical cells according to embodiments disclosed herein having electrolyte material including 2% VC is shown in FIG. 6B. The horizontal axis represents the electrochemical cells formed, including cells with control electrolyte materials having no electrolyte additive (LCO1_no additive), illustrative electrochemical cells with electrolyte materials having 2% VC (LCO1_2% VC and LCO2_2% VC), and four electrochemical cells with comparative electrolyte additives (LCO2_HVE1, LCO2_HVE2, LCO3_HVE3, and LCO3_HVE4). The vertical axis represents the amount of swelling (as a percentage increase in thickness measured at the center of the cell compared with the initial cell thickness). As shown in FIG. 6B, swelling in the cell with control electrolyte material having no high-voltage additive increased the cell thickness by more than 5%. the illustrative cells with 2% VC electrolyte material did not swell and no pressure release (e.g., burping) was needed. Additionally, while cell LCO3_HVE4 was observed in the accelerated stability studies to undergo at least 105 days of 75° C. accelerated daily rate cycling before the reaching 90% capacity retention, swelling in cell LCO3_HVE4 increased the cell's thickness by about 1% or greater.

### Example Analysis—LCO Utilization and Electrolyte Material With VC Additive

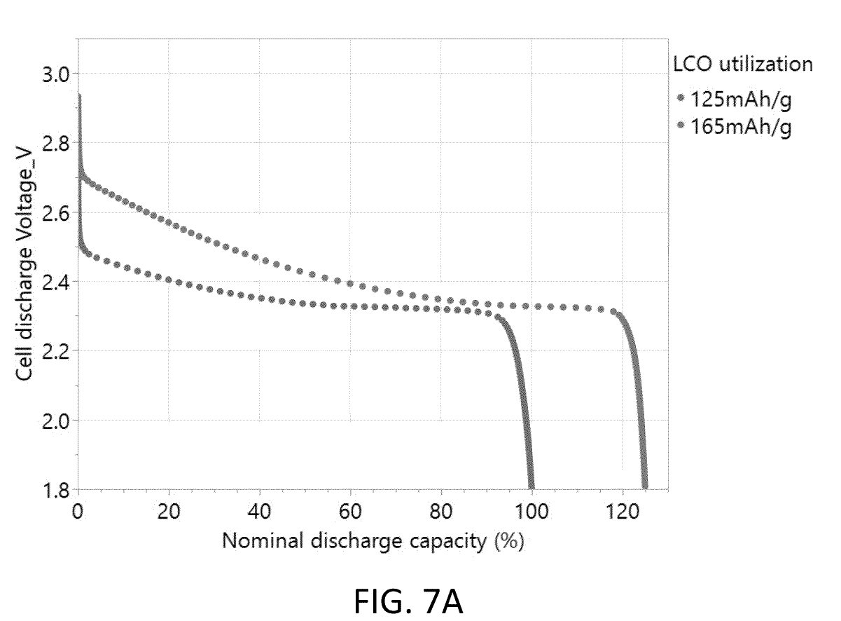

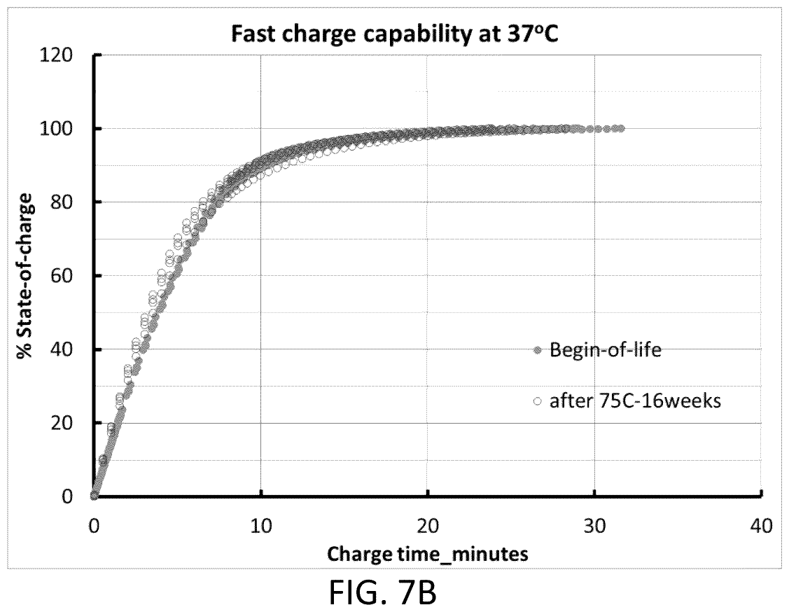

As discussed herein, illustrative electrochemical cells with relatively high LCO utilization and an electrolyte material including VC may advantageously improve cell energy density. A graphical representation of the voltage discharge curve of an illustrative electrochemical cell with relatively high LCO utilization and an electrolyte material including VC is shown in FIG. 7A. The illustrative electrochemical cell had an LCO utilization of 165 mAh/g, an electrolyte material including 2% VC, and an upper recharge voltage of 3 V. An electrochemical cell with a comparative LCO utilization of 125 mAh/g, an electrolyte material without VC, and an upper recharge voltage of 2.8 V was also tested. The horizontal axis represents the nominal discharge capacity of each cell relative to the nominal discharge capacity of the comparative cell. The vertical axis represents each cell's discharge voltage. As shown in FIG. 7A, the illustrative electrochemical cell with high LCO utilization and a VC electrolyte additive afforded a more than 20% improvement in energy density compared with the comparative cell.

As also discussed herein, while high LCO utilization typically results in reduced rate retention over time, illustrative electrochemical cells with relatively high LCO utilization and an electrolyte material including VC may advantageously show improved rate retention over time. A graphical representation of charge rate (at 37° C. ambient temperature) before and after 16 weeks of 75° C. accelerated storage stability testing is shown in FIG. 7B for the illustrative electrochemical cells with relatively high LCO utilization and an electrolyte material including VC. For rate retention, the 75° C. accelerated storage stability test provides an acceleration factor of 67.5 compared to storage at 37° C. (i.e., ambient body temperature). In other words, for rate retention, 16 weeks of accelerated storage stability testing at 75° C. is equivalent to 20 years of storage at 37° C. In accelerated storage stability testing, each cell was fully charged and then kept in open circuit storage at 75° C. ambient temperature for 16 weeks. Every two weeks, each cell underwent a full characterization cycle at 37° C., and then fully recharged before being returned to the open circuit storage at 75° C. Accelerated storage stability testing may be useful to evaluate medical device applications (e.g., implantable medical device applications), wherein a device may be used for a very long service life (e.g., 15 years or greater) and may undergo relatively few charge/discharge cycles during that service life. Furthermore, in implantable medical device applications, a cell may primarily undergo partial charge/discharge cycles, for example, only discharging from 100% to 80% before being recharged to 100%.

The vertical axis represents the state-of-charge (as a percentage, where 100% represents fully charged). The horizontal axis represents the time elapsed during charging (in minutes). As shown in FIG. 7B, each illustrative cell demonstrates stable fast recharge capability over time, and each illustrative cell can be recharged to 95% or greater within 20 minutes both before and after 16 weeks of 75° C. accelerated storage stability testing.

What is claimed is:

1. An implantable medical device comprising a rechargeable lithium-ion cell comprising:
a negative electrode comprising a negative electrode active material comprising LTO;
a positive electrode comprising a positive electrode active material comprising LCO;
a separator between the negative electrode and the positive electrode; and
an electrolyte material comprising from 0.5 to 5 percent by weight vinylene carbonate as a high-voltage additive, wherein the cell has a cell upper recharge voltage of between 2.6 V and 3.4 V, and wherein at least one of:
the negative electrode has a negative electrode area specific capacity between 1 mAh/cm² and 15 mAh/cm², or
the positive electrode has a LCO utilization rate between about 120 mAh/g and about 220 mAh/g.

2. The implantable medical device of claim 1, wherein the LCO of the positive electrode active material comprises particles having a multi-modal size distribution.

3. The implantable medical device of claim 2, wherein the LCO of the positive electrode active material comprises particles having a first average particle size and particles having a second average particle size; and wherein the first average particle size is between 10 um and 30 um and the second average particle size is between 2 um and 8 um.

4. The implantable medical device of claim 1, wherein the positive electrode further comprises a binder comprising polyvinylidene fluoride (PVDF).

5. The implantable medical device of claim 1, wherein the positive electrode further comprises a carbon conductive agent comprising carbon black, graphite, or a combination thereof.

6. The implantable medical device of claim 1, wherein the positive electrode has a porosity between 15% and 25%.

7. The implantable medical device of claim 1, wherein the negative electrode has a porosity of between 20% and 50%.

8. The implantable medical device of claim 1, wherein the negative electrode has a thickness between 0.1 mm and 0.2 mm.

9. The implantable medical device of claim 1, wherein the negative electrode has an active material loading value between 6 mg/cm² and 60 mg/cm².

10. The implantable medical device of claim 1, wherein the negative electrode has an LTO utilization between 150 mAh/g and 180 mAh/g.

11. The implantable medical device of claim 1, wherein the negative electrode comprises an LTO content between 85% and 98%.

12. The implantable medical device of claim 1, wherein the positive electrode comprises an LCO content between 90% and 99%.

13. The implantable medical device of claim 1, wherein the positive electrode has a density between 3 g/cm³ and 4.2 g/cm³.

14. The implantable medical device of claim 1, wherein the positive electrode has a positive electrode area specific capacity, and wherein a N/P capacity ratio of the negative electrode area specific capacity to the positive electrode area specific capacity is between 0.5:1 and 1:1.

15. The implantable medical device of claim 1, wherein the cell comprises an electrolyte content between 2 g/Ah and 10 g/Ah.

16. The implantable medical device of claim 1, wherein the cell can be charged at a charge rate between C/100 and C/0.1.
